(19) United States
(12) Patent Application Publication
(10) Pub. No.: US 2006/0149064 A1
(43) Pub. Date: Jul. 6, 2006

(54) Process to prepare malayamycin derivatives

(76) Inventors: Stephen Hanessian, Montreal (CA); Roger Machaalani, Montreal (CA); Stephane Michael Jean Marcotte, Mont Saint Aignan (FR)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/544,660

(22) PCT Filed: Jan. 28, 2004
(86) PCT No.: PCT/GB04/00359

§ 371 Date: Aug. 4, 2005

(30) Foreign Application Priority Data
Feb. 5, 2003 (GB) 0302648.1

(51) Int. Cl. C07D 493/02

(52) U.S. Cl. 544/310

(57) ABSTRACT

A process is provided for the preparation of a compound of the general formula (I): wherein R is H or C₁₋₄ alkyl, which comprises treating a compound of the general formula (II): where R″ is R or R⁸CO, R is as defined above, R⁸ is C₁₋₈ alkyl or optionally substituted phenyl and R⁹ is optionally substituted C₁₋₈ alkyl or optionally substituted aryl, with an amine R′″NH₂ wherein R′″ is H or C₁₋₄ alkyl. Also provided are the trans isomers of the compound (I) where R is CH₃ (6-epi-malayamycin A) and H (6-epi-desmethylmalayamycin A), the cis and trans isomers of the compound (I) where R is C₂₋₄alkyl and various intermediate compounds.

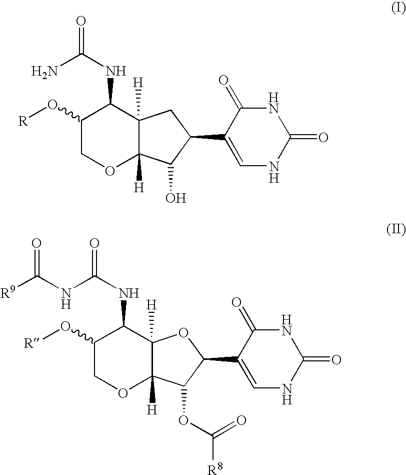

This invention relates to a process for the preparation of biocidal compounds. More particularly it relates to a synthetic method for preparing biocidal compounds, certain of which are novel and certain of which have only previously been obtained by cultivation of Streptomyces organisms. It also relates to novel intermediates used in the process.

Biocidal compounds of the formula (A): 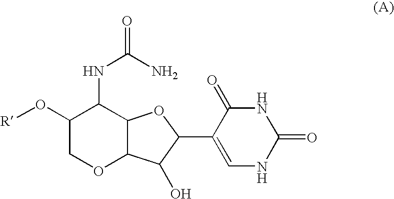
where R′ is H or CH₃, and their isolation from a fermentation broth of a strain of micro-organism from the species Streptomyces malaysiensis are described in International patent application No. PCT/GB2003/000063. The compounds of formula (A) have six asymmetric centres and may exist in the form of one or more isomers. Particularly mentioned in PCT/GB2003/000063 are the compounds of formulae (B) and (C), which are named malayamycin A and desmethylmalayamycin A, respectively.

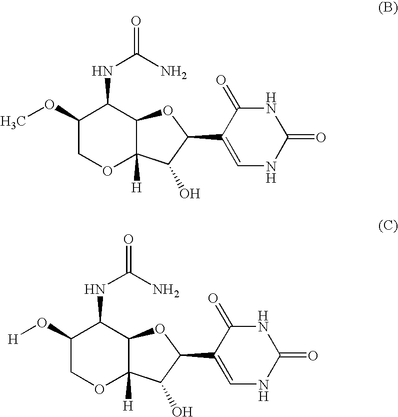

These compounds are biocidal agents, showing antiviral and anti-cancer properties. They are, however, of particular interest as antifungal agents, especially against plant pathogenic fungi.

The present invention provides a synthetic route to malayamycin A and desmethylmalayamycin A and to certain of their isomers and analogues.

Thus according to the present invention there is provided a process for the preparation of a compound of the general formula (I): 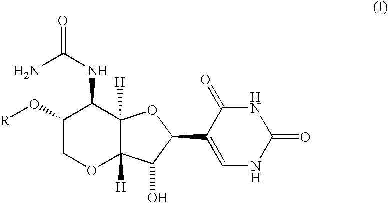
wherein R is H or C₁₋₄ alkyl (especially methyl), which comprises treating a compound of the general formula (II): 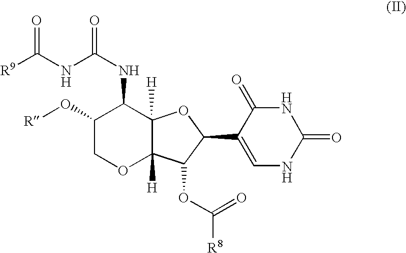
wherein R″ is R or R⁸CO, R is as defined above, R⁸ is C₁₋₈ alkyl or optionally substituted phenyl and R⁹ is optionally substituted C₁₋₈ alkyl or optionally substituted aryl, with an amine R′″NH₂ wherein R′″ is H or C₁₋₄ alkyl.

In the compound of general formula (I), the RO group may be cis or trans to the NH₂CONH group. The cis isomer of the compound where R is CH₃ is malayamycin A and the cis isomer of the compound where R is H is desmethylmalayamycin A. The trans isomer of the compound where R is CH₃ (6-epi-malayamycin A) and the trans isomer of the compound where R is H (6-epi-desmethylmalayamycin A) are novel compounds and form a further part of this invention as to do both the cis and trans isomers of the compounds where R is C₂₋₄ alkyl.

Throughout this specification, the number of carbon atoms that alkyl moieties may contain is usually stated. Where unstated, alkyl moieties may contain from 1 to 8, suitably from 1 to 6 and typically from 1 to 4, carbon atoms. In all cases they may be in the form of straight or branched chains. Examples are methyl, ethyl, n- and iso-propyl, n-, sec-, iso- and tert-butyl, n-pentyl and n-hexyl. Suitable optional substituents of alkyl moieties include halo (e.g. chloro, bromo and fluoro), C₁₋₆ alkoxy and C₁₋₆ alkylthio.

Aryl is usually phenyl. Optional substituents of aryl and aryl moieties such as benzyl include C₁₋₆ alkyl, C₂₋₆ alkenyl, C₂₋₆ alkynyl, C₁₋₆ alkoxy, C₂₋₆ alkenyloxy, C₂₋₄ alkynyloxy, halo(C₁₋₆)alkyl, halo(C₁₋₆)alkoxy, C₁₋₆ alkylthio, halo(C₁₋₆)alkylthio, C₁₋₄ alkoxy(C₁₋₆)alkyl, C₃₋₆ cycloalkyl, C₃₋₆ cycloalkyl(C₁₋₄)alkyl, phenoxy, benzyloxy, benzoyloxy, cyano, nitro, —CONR^{a}R^{b}, —SO₂R^{a}, —OSO₂R^{a}, —COR^{a}, —CR^{a}═NR^{b} or —N═CR^{a}R^{b}, in which R^{a} and R^{b} are independently hydrogen, C₁₋₄ alkyl, halo(C₁₋₄)alkyl, C₁₋₄ alkoxy, halo(C₁₋₄)alkoxy, C₁₋₄ alkyl-thio, C₃₋₆ cycloalkyl, C₃₋₆ cycloalkyl(C₁₋₄)alkyl, phenyl or benzyl.

R⁸ is typically tert-butyl and R⁹ is typically C₁₋₄ alkyl, e.g. methyl, substituted with halo. A particular example of R⁹ is trichloromethyl.

In one aspect the invention includes a process as defined above wherein R is H or C₁₋₄ alkyl, R″ is R or R⁸CO, R⁸ is C₁₋₆ alkyl and R⁹ is C₁₋₆ alkyl substituted with halo, especially chloro.

In another aspect the invention includes a process wherein R is H or methyl, R″ is methyl or R⁸CO, R⁸ is tert-butyl and R⁹ is trichloromethyl.

In more detail, the compounds of the invention can be made as shown in Schemes 1 to 6.

The starting materials for the synthesis are either readily available commercially, for example ribonolactone (i.e. D-(+)-ribonic γ-lactone), or can be made by methods known in the literature, for example 2,4-dimethoxypyrimidine (Gilbert, G. E.; Johnson, T. B. J. Am. Chem. Soc. 1930, 52, 2001) and 2,4-dimethoxy-5-iodopyrimidine (Das, B.; Kundu, N. G. Synthetic Comm. 1988, 18, 855).

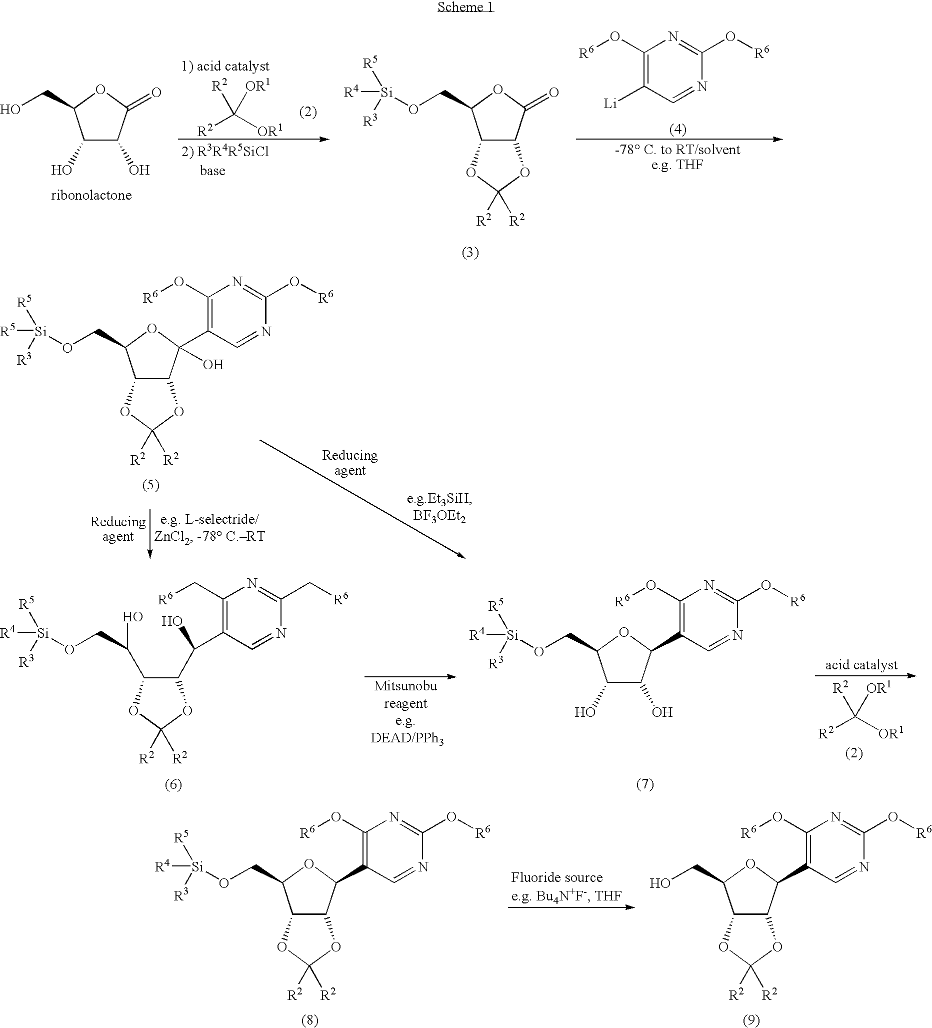

In Scheme 1 ribonolactone can be converted into compounds of general formula (3), by reaction with ketals of general formula (2), where R¹ and R² are C₁₋₄ alkyl, and with catalysis by an acid such p-toluene sulphonic acid, followed by a silyl chloride of general formula R³R⁴R⁵SiCl, where R³, R⁴, and R⁵ can be independently C₁₋₄ alkyl, or phenyl, in the presence of a suitable base such as imidazole. Compounds of general formula (5) can be formed by reaction of compounds of general formula (3) with compounds of general formula (4) at a temperature between −78° C. and −30° C., but preferably at −78° C., in a suitable solvent such as tetrahydrofuran (THF). Compounds of general formula (4) can be generated by treatment of 5-bromo- or 5-iodo-2,4-dialkoxypyrimidine, where the alkyl group R⁶ is C₁₋₄ alkyl, with n-, s- or t-butyl lithium. Compounds of general formula (5) can exist as either the beta- or alpha-anomer, or as a mixture. Compounds of general formula (7) can be formed from compounds of general formula (5) by reduction with a suitable reducing agent such as triethylsilane, in the presence of a Lewis acid such as boron trifluoride etherate, in a suitable solvent such as dichloromethane (DCM), followed by chromatographic separation of the mixture of anomers. Alternatively compounds of general formula (5) can be first selectively reduced to compounds of general formula (6), by reaction with a reducing agent such as L-selectride, in the presence of a Lewis acid such as zinc chloride, in a suitable solvent such as DCM, at a temperature starting at −78° C. and warming to room temperature. Compounds of general formula (6) can then be reacted under Mitsunobu conditions, for example with diethylazodicarboxylate (DEAD) and triphenyl phosphine, in a suitable solvent such as DCM. Compounds of general formula (8) can be formed by reaction of compounds of general formula (7) with compounds of general formula (2), with catalysis by an acid such p-toluene sulphonic acid. Compounds of general formula (9) can be formed by de-silylation of compounds of general formula (8), for example with a source of fluoride ion such as tetrabutylammonium fluoride, in a suitable solvent such as THF.

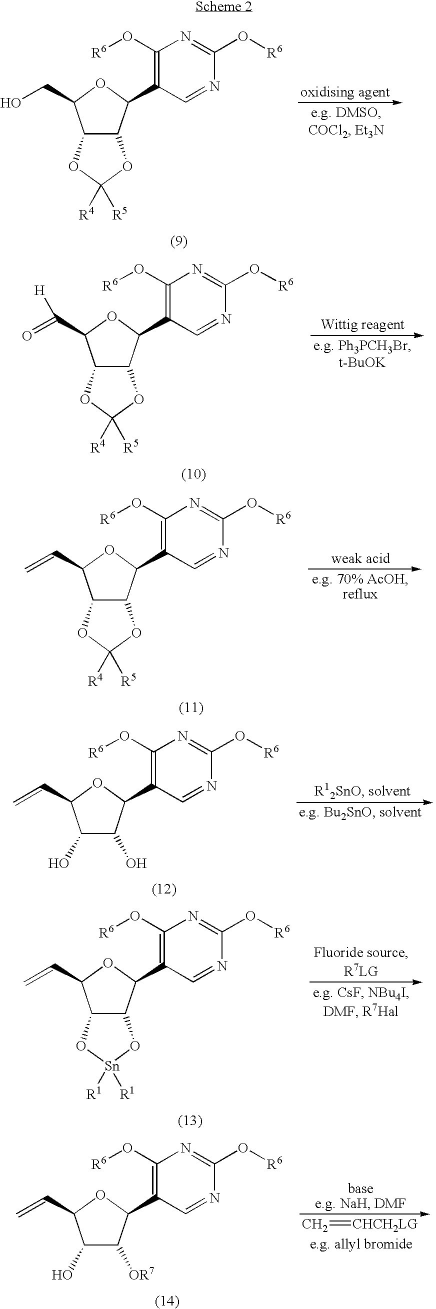

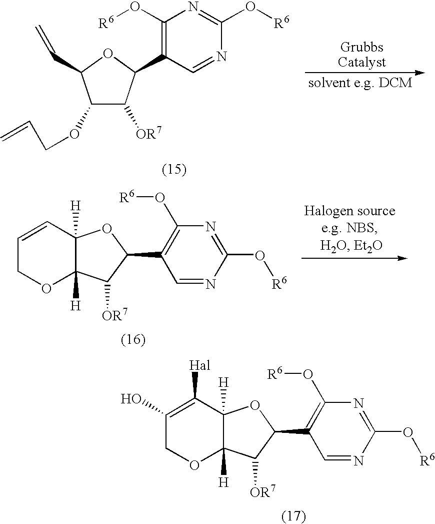

In Scheme 2 compounds of general formula (10) can be formed by oxidising compounds of general formula (9) with a suitable oxidising agent such as dimethyl-sulphoxide and oxalyl chloride in the presence of triethylamine. Compounds of general formula (11) can be formed by treatment of compounds of general formula (10) with a Wittig reagent such as methylenetriphenylphosphorane, generated by reacting a methyltriphenylphosphonium salt with a strong base such as potassium t-butoxide. Compounds of general formula (12) can be formed by deprotection of compounds of general formula (11) by treatment with a weak acid such as 70% acetic acid. Compounds of general formula (13) can be formed by reacting compounds of general formula (12) with a trialkyltin oxide of general formula R¹₂SnO, where R¹ is as defined above. Compounds of general formula (14) can be formed by reaction of compounds of general formula (13) with a source of fluoride ion such as caesium fluoride, in the presence of an alkylating agent R⁷LG, where R⁷ is a substituted benzyl group, such as 4-methoxybenzyl, and LG is a leaving group such as chlorine or bromine. Compounds of general formula (15) can be formed from compounds of general formula (14) by reaction with an allylating agent CH₂═CHCH₂LG, such as allyl bromide, in the presence of a base such as sodium hydride, in a suitable solvent such as dimethylformamide (DMF). Compounds of general formula (16) can be formed from compounds of general formula (15) in a ring-closing metathesis reaction by treatment with the Grubbs catalyst, [(cyclohexyl)₃P]₂Cl₂Ru═CHPh where Ph is phenyl, in a suitable solvent such as DCM, at a temperature between room temperature and 40° C. Compounds of general formula (17), where Hal is a halogen atom such as chlorine, bromine or iodine, can be formed by reaction of compounds of general formula (16) with a halogen source such as N-bromosuccinimide, in the presence of water and an organic solvent such as diethyl ether.

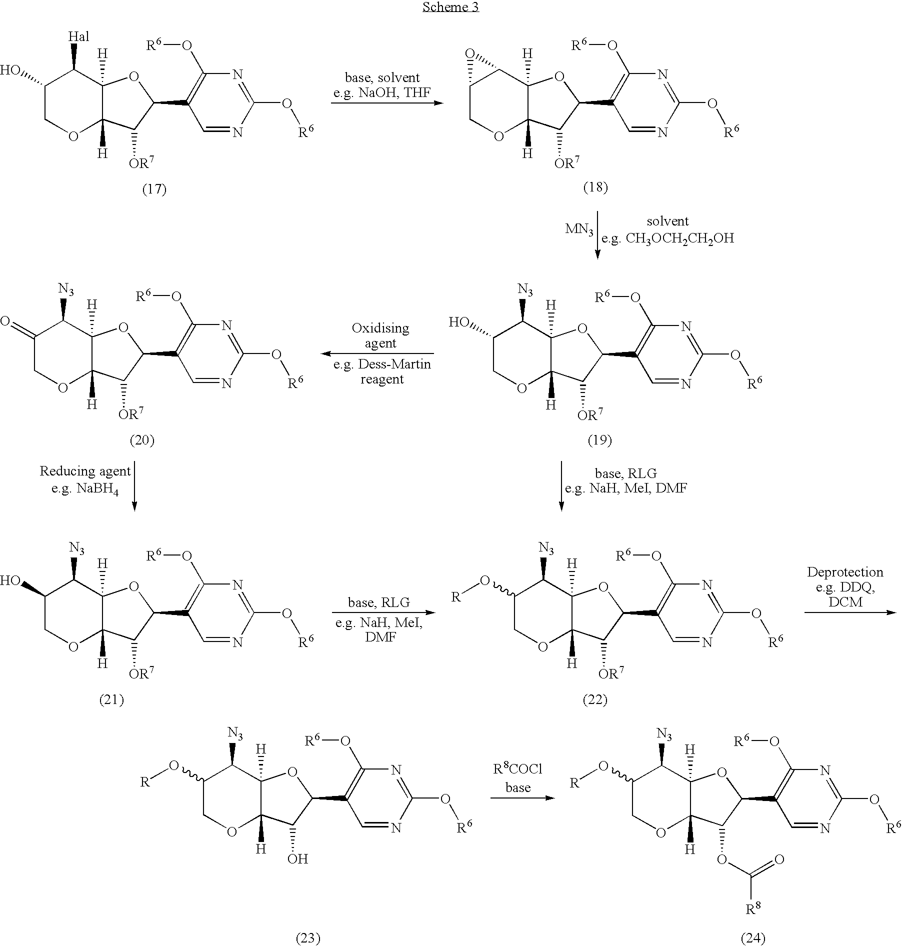

In Scheme 3 compounds of general formula (18) can be formed from olefins of general formula (17) by reaction with a base such as sodium hydroxide in a suitable solvent such as THF. Compounds of general formula (19) can be formed by treatment of compounds of general formula (18) with a metal azide MN₃ where M is for example an alkali metal, such as sodium azide, in a suitable solvent such as methoxyethanol.

Compounds of general formula (22) where the RO group, in which R is C₁₋₄ alkyl, is cis to the azide group, can be formed from compounds of general formula (19) in three steps. Compounds of general formula (20) can be formed from compounds of general formula (19) by oxidation with a suitable oxidising agent such as [1,1,1-tris(acetyloxy)-1,1-dihydro-1,2-benzodioxol-3-(1H)-one] (Dess-Martin periodinane) in a suitable solvent such as DCM. Compounds of general formula (21) can be formed from compounds of general formula (20) by reduction with a suitable reducing agent such as sodium borohydride, in a suitable solvent such as methanol. Compounds of general formula (22), where the RO group is cis to the azide group, can be formed by reaction of compounds of general formula (21) with a base such as sodium hydride, and a compound of general formula RLG, where R is C₁₋₄ alkyl and LG is a leaving group. An example of RLG is methyl iodide.

Compounds of general formula (22) where the RO group is trans to the azide group, can be formed by reaction of compounds of general formula (19) directly with a base such as sodium hydride, and a compound of general formula RLG, such as methyl iodide. Compounds of general formula (23), where the RO group can be either cis or trans to the azide group, can be formed from compounds of general formula (22) by deprotection with a reagent such as 2,3-dichloro-5,6-dicyano-1,4-benzoquinone (DDQ). Compounds of general formula (24), where the RO group can be either cis or trans to the azide group, can be formed from alcohols of general formula (23) by reaction with acid chlorides of general formula R⁸COCl, where R⁸ is a C₁₋₈ alkyl or optionally substituted phenyl group, in the presence of a base such as triethylamine and a basic catalyst such as 4-dimethylamino-pyridine (DMAP), in a suitable solvent such as pyridine.

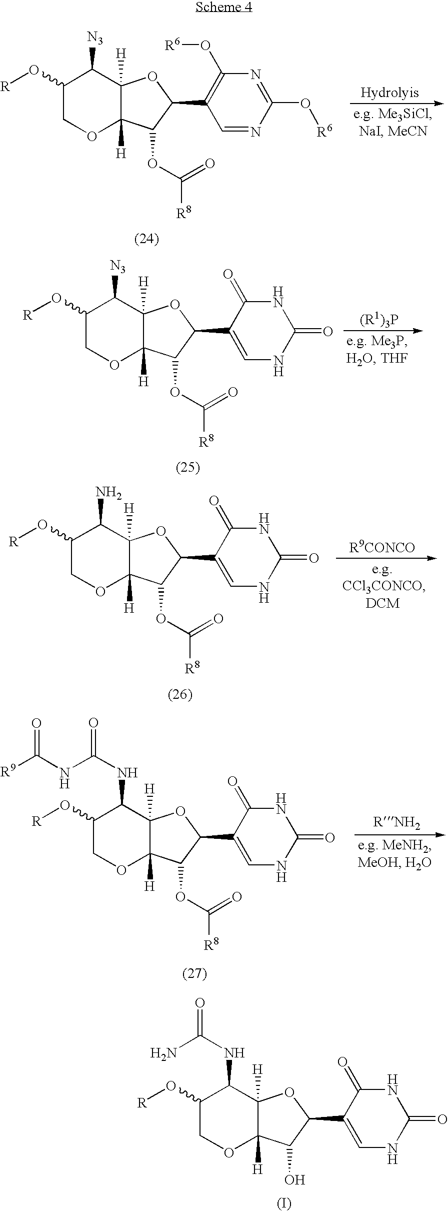

In Scheme 4 compounds of general formula (25), where the RO group can be either cis or trans to the azide group, can be formed from compounds of general formula (24) by reaction with a deprotecting reagent such as trimethylsilyl chloride and sodium iodide in a suitable solvent such as acetonitrile. Compounds of general formula (26) where the RO group can be either cis or trans to the amino group can be formed by reaction of compounds of general formula (25) with a reducing agent such as a trialkylphosphine (R¹)₃P, where R¹ is as defined above, for example trimethylphosphine, in a suitable solvent such as a mixture of water and THF. Compounds of general formula (27), where the RO group is cis or trans to the R⁹CONHCONH group can be formed by reacting compounds of general formula (26) with an isocyanate R⁹CONCO, where R⁹ can be C₁₋₈ alkyl group optionally substituted with a halo group such as chlorine or bromine, for example trichloroacetyl isocyanate, or an optionally substituted aryl group, in a suitable solvent such as DCM.

Compounds of general formula (1) can be made from compounds of general formula (27), where the RO group is cis or trans to the NH₂CONH group, can be made by deprotection with an amine R′″NH₂, where R′″ is as defined above, in a suitable solvent such as a mixture of methanol and water.

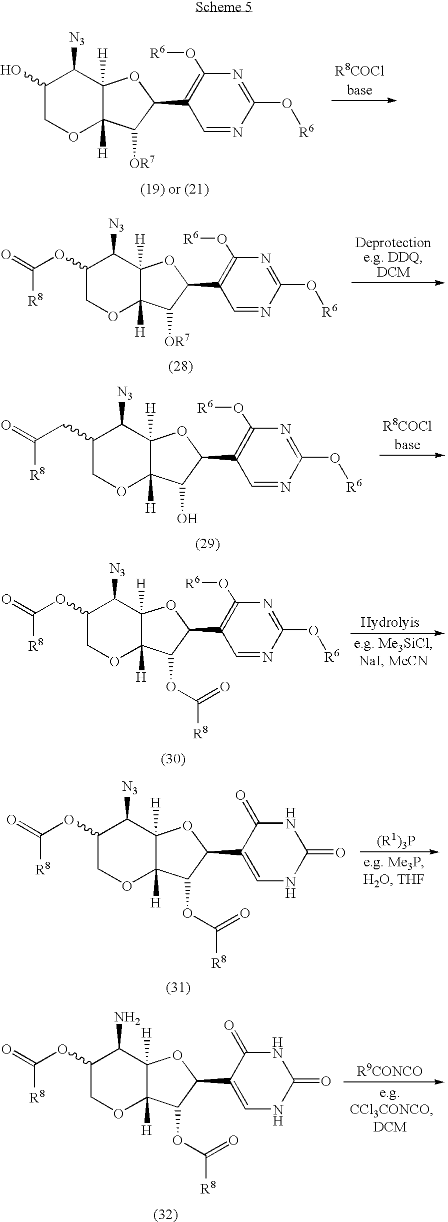

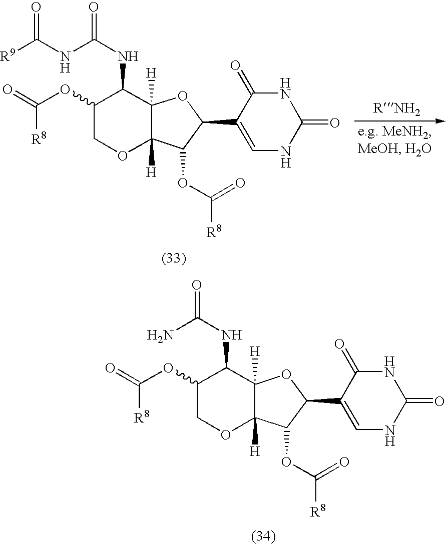

Scheme 5 shows the preparation of compounds of general formula (34), which are particular examples of compounds of general formula (I), where R is H.

Compounds of general formula (28) can be prepared from alcohols of general formula (19) or (21) by reaction with acid chlorides of general formula R⁸COCl, in the presence of a base such as triethylamine, and a basic catalyst such DMAP. Compounds of general formula (29) where the R⁸COO group can be cis or trans to the azide group, can be formed from compounds of general formula (28) by deprotection with a reagent such as DDQ, in a suitable solvent such as DCM. Compounds of general formula (30), where the R⁸COO group can be cis or trans to the azide group, can be formed from compounds of general formula (29) by reaction with acid chlorides of general formula R⁸COCl, in the presence of a base such as triethylamine, and a basic catalyst such as DMAP. Compounds of general formula (31), where the R⁸COO group can be cis or trans to the azide group, can be formed from compounds of general formula (30) by reaction with a deprotecting reagent such as trimethylsilyl chloride and sodium iodide in a suitable solvent such as acetonitrile. Compounds of general formula (32), where the R⁸COO group can be cis or trans to the amine group, can be formed by reaction of compounds of general formula (31) with a reducing agent such as a trialkylphosphine (R¹)₃P, where R¹ is as defined above, for example trimethylphosphine, in a suitable solvent such as a mixture of water and THF. Compounds of general formula (33), where the R⁸COO group can be cis or trans to the R⁹CONHCONH group can be formed by reacting compounds of general formula (32) with an isocyanate R⁹CONCO, where R⁹ can be C₁₋₈ alkyl group optionally substituted with a halo group such as chlorine or bromine, for example trichloroacetyl isocyanate, or an optionally substituted aryl group, in a suitable solvent such as DCM.

Compounds of general formula (34) can be made from compounds of general formula (33), where the R⁸COO group is cis or trans to the NH₂CONH group, by deprotection with an amine R′″NH₂, for example methylamine, where R′″ is as defined above, in a suitable solvent such as a mixture of methanol and water.

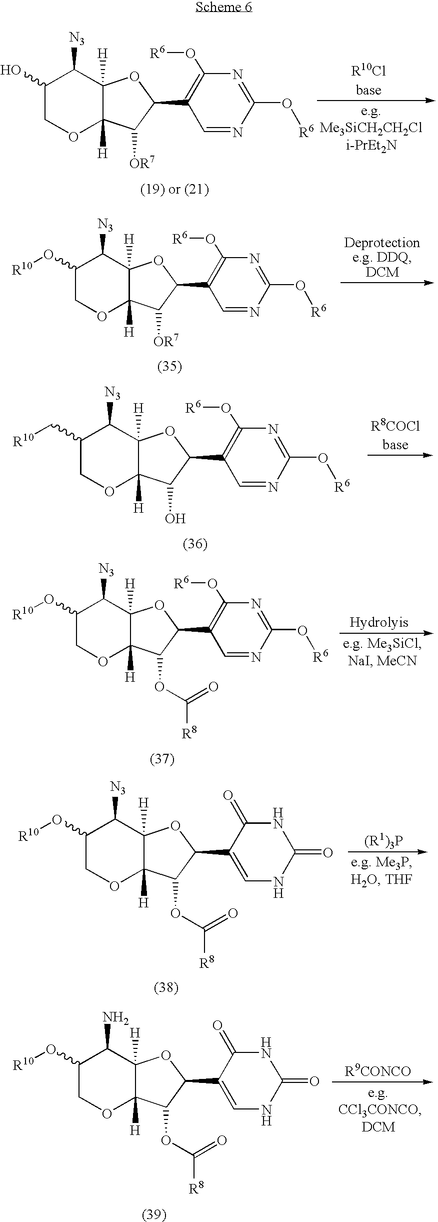

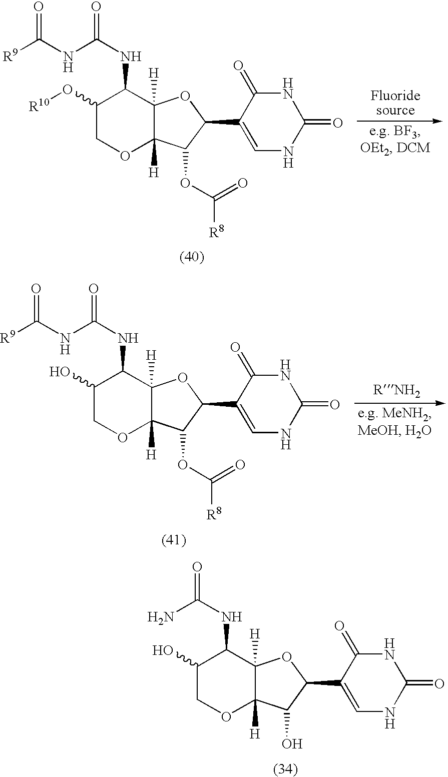

Scheme 6 shows the preparation of compounds of general formula (34), which are particular examples of compounds of general formula (I), where R is H.

Compounds of general formula (35), where R¹⁰ is a suitable protecting group such as a (R¹¹)₃SiCH₂CH₂OCH₂ group where R¹¹ is C₁₋₄ alkyl, can be prepared from alcohols of general formula (19) or (21) by reaction with halides of general formula R¹⁰Hal, where Hal is chlorine or bromine, in the presence of a base such as iso-propyldiethylamine, at between room temperature and 80° C., but preferably between 30° C. and 50° C., in a suitable solvent such as DCM. Compounds of general formula (36) where the R¹⁰O can be cis or trans to the azide group, can be formed from compounds of general formula (35) by deprotection with a reagent such as DDQ, in a suitable solvent such as DCM. Compounds of general formula (37), where the R¹⁰O group can be cis or trans to the azide group, can be formed from compounds of general formula (36) by reaction with acid chlorides of general formula R⁸COCl, in the presence of a base such as triethylamine, and a basic catalyst such as DMAP. Compounds of general formula (38), where the R¹⁰O group can be cis or trans to the azide group, can be formed from compounds of general formula (37) by reaction with a deprotecting reagent such as trimethylsilyl chloride and sodium iodide in a suitable solvent such as acetonitrile. Compounds of general formula (39), where the R¹⁰O group can be cis or trans to the amine group, can be formed by reaction of compounds of general formula (38) with a reducing agent such as a trialkylphosphine (R¹)₃P, where R¹ is as defined above, for example trimethylphosphine, in a suitable solvent such as a mixture of water and THF. Compounds of general formula (40), where the R¹⁰O group can be cis or trans to the R⁹CONHCONH group can be formed by reacting compounds of general formula (39) with an isocyanate R⁹CONCO, where R⁹ can be C₁₋₈ alkyl group optionally substituted with a halo group such as chlorine or bromine, for example trichloroacetyl isocyanate, or an optionally substituted aryl group, in a suitable solvent such as DCM. Compounds of general formula (41) can be made from compounds of general formula (40), by reaction with a source of fluoride ion such as boron trifluoride etherate, between 0° C. and room temperature, in a suitable solvent such as DCM.

Compounds of general formula (34) can be made from compounds of general formula (41), where the OH group is cis or trans to the NH₂CONH group, by deprotection with an amine R′″NH₂, for example methylamine, where R′″ is as defined above, in a suitable solvent such as a mixture of methanol and water

In a further aspect the invention includes the intermediate compounds (16) to (33) and (35) to (41) as defined above.

Compounds that may be made by the process of the invention are illustrated in Table 1. The compounds in Table 1 have the general formula (I) in which R and the orientation of RO to the NH₂CONH— group are as shown in the table. “Me” represents “methyl”.

TABLE 1

| | |
|---|---|
| | (I) |
| 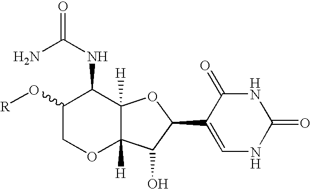 | |

| Compound | | Orientation of | |
|---|---|---|---|
| No. | R | RO to NH₂CONH— | Structure |
| 1 | Me | cis | 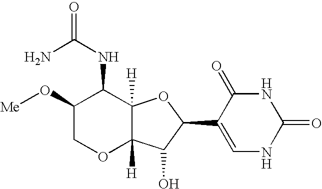 |
| 2 | Me | trans | 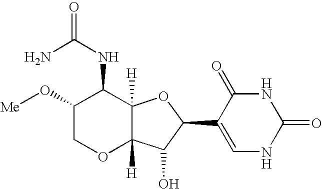 |
| 3 | H | cis | 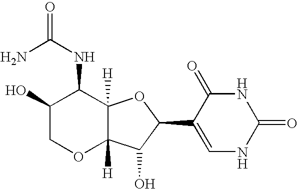 |
| 4 | H | trans | 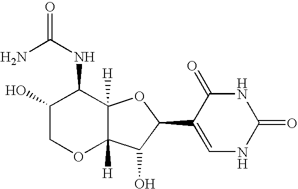 |

The compounds Nos. 2 and 4 of Table 1 are novel and form a part of the present invention. The invention is further illustrated by the following Examples, in which, in the structural formulae, Me is methyl, t-Bu is tertiary butyl, Ph is phenyl, PMB is p-methoxybenzyl, DMF is N,N-dimethylformamide, THF is tetrahydrofuran, DMSO is dimethyl sulphoxide, DCM is dichloromethane, DDQ is 2,3-dichloro-5,6-dicycano-1,4-benzoquinone and DMAP is 4-dimethylaminopyridine.

### EXAMPLE 1

This Example illustrates the preparation of malayamycin A (Compound No. 1 of Table 1).

### Step 1

The preparation of: 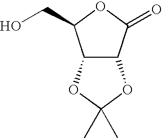
D-(+)-ribonolactone (4.35 g, 29.3 mmol), 2,2-dimethoxypropane (18.1 ml, 146 mmol) and pyridinium p-toluene sulfonate (195 mg, 0.79 mmol) were mixed together. The light yellow mixture was heated at 60° C. for 4 hours, and concentrated. The yellow oil was taken up in ethyl acetate, washed with saturated sodium bicarbonate and with brine. The organic layer was dried with magnesium sulphate and concentrated to give a white solid, which was dissolved in THF and a solution of 0.1 M hydrochloric acid (10 ml) was added. The colourless mixture was stirred for 10 minutes at room temperature, concentrated to half volume, and diluted with ethyl acetate. The combined organic phases were washed with saturated sodium bicarbonate and brine, dried over magnesium sulphate and evaporated to give the desired product as a white solid, which was recrystallized from 1:1 hexane:ethyl acetate, (3.5 g, 72%).

[α]_{D}²⁰=+70.5 (c 1, CHCl₃) ¹H NMR (CDCl₃, 300 MHz) δ ppm: δ 1.40 (s, 3H, CH₃), 1.50 (s, 3H, CH₃), 2.60 (t, 1H, OH), 3.72 (dd, 1H, H-5), 4.71 (dd, 1H, H-5), 4.71 (t, 1H, H-3), 4.81 (d, 1H, H-4), 4.90 (d, 1H, H-2). ¹³C NMR (CDCl₃, 300 MHz) δ ppm: 25.10, 26.09, 62.18, 76.12, 78.30, 84.20, 113.98, 175.16.

### Step 2

The preparation of: 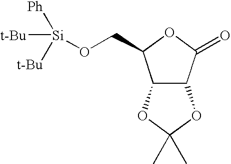
The product of step 1 (3.25 g, 17 mmol), imidazole (2.25 g, 37.4 mmol) and DMF (anhydrous, 25 ml) were mixed and then t-butyldiphenylsilyl chloride (4.6 g, 17 mmol) was added dropwise. The colourless mixture was stirred at room temperature for 24 hours, and then poured into cold water. The aqueous layer was extracted with ether, the combined organic phase was dried with magnesium sulphate, evaporated to give the desired product as a white solid, which was recrystallized from hexane (7.02 g, 97%), m.p. 86-88° C.

[α]_{D}²⁰=+10.7 (c 1, CHCl₃) ¹H NMR (CDCl₃, 400 MHz) δ ppm: 1.06 (9H, s, tBu), 1.41 (s, 3H, CH₃), 1.49 (s, 3H, CH₃), 3.77 (dd, 1H, J=1.3, J=11.4, H-5), 3.93 (dd, 1H, J=2.2, J=11.51, H-5), 4.59 (s, 1H,), 4.75 (d, 1H, J=5.6), 4.91 (d, 1H, J=6.2), 7.4-7.6 (6H, Ph), 7.6-7.65 (4H, Ph). ¹³C NMR (CDCl₃, 400 MHz) δ ppm: 14.53, 23.06, 26.01, 27.17, 63.97, 76.25, 78.86, 82.73, 113.55, 128.42, 128.44, 130.60, 130.61, 131.95, 132.75, 135.85, 136.02, 174.51.

### Step 3

The preparation of: 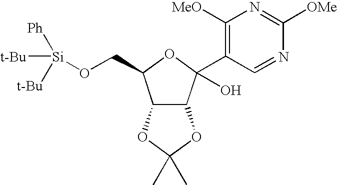
2,4-Dimethoxy-5-iodopyrimidine (1.53 g, 5.80 mmol) was dissolved in dry THF (55 ml) under an argon atmosphere, and tert-butyl lithium (6.73 ml, 11.44 mmol, 1.7 M in hexane) was added dropwise over five minutes at −78° C. and the reaction mixture was stirred for 30 minutes. The product of step 2 (2.25 g, 5.28 mmol) dissolved in dry THF (57 ml) was slowly added to the lithium reagent over 10 minutes. The mixture was stirred at −78° C. for four hours, and then at −20° C. for one hour. Brine was added, the organic phase was separated, extracted with diethyl ether, and the combined organic phases dried over magnesium sulphate. The organic fraction was evaporated to afford an oil which was purified by flash chromatography over silica eluting with 8:2 hexane:ethyl acetate to give the (1.78 g, 3.16 mmol, 60%) as a white solid, which was shown to be a 1:8 mixture of the alpha:beta anomers, with the beta-anomer being the desired product.
Beta anomer:

M.p 51° C., [α]_{D}²⁰=−45.3 (c 1, CHCl₃) ¹H NMR (CDCl₃, 400 MHz) δ ppm: 1.08 (s, 9H, tBu), 1.17 (s, 3H, CH₃), 1.24 (s, 3H, CH₃), 3.98 (s, 3H, OMe), 4.02 (s, 3H, OMe), 4.22 (s, 1H, OH-1′), 4.35 (m, 1H, H-4′), 4.84 (m, 2H, H-3′, H-2′), 7.24-7.5 (m, 6H, Ph), 7.51-7.7 (m, 4H, Ph), 8.47 (s, 1H, H-6).

### Step 4

The preparation of: 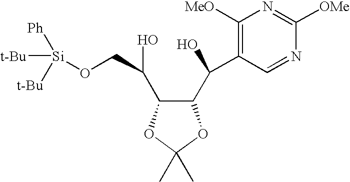
The product of step 3, (1 g, 1.77 mmol) was dissolved in anhydrous dichloromethane (100 ml) under an argon atmosphere. The colourless solution was brought to −78° C. and zinc chloride (5.31 ml, 5.31 mmol, 1 M in diethyl ether) was added. The colourless mixture was stirred at −78° C. for 30 minutes and L-selectride (17.7 ml, 17.7 mmol, 1 M in THF) was added dropwise very slowly over 10 minutes at −78° C. The colourless mixture was stirred overnight (−78° C. to room temperature slowly), and after 18 hours, water (5 ml) was added to the yellow mixture, which was stirred until it came colourless. After addition of 95% ethanol (15 ml), a solution of sodium hydroxide (9 ml, 6 M) was added and the mixture was cooled to 0° C. before 30% hydrogen peroxide (15 ml) was added dropwise. The mixture was stirred for 10 minutes at 0° C., then at room temperature for 5 minutes. The organic phase was separated and the aqueous phase was saturated with solid potassium carbonate (1 g). The aqueous layer was extracted with ethyl acetate and the combined organic phase was dried over magnesium sulphate. The organic fraction was evaporated to give a colourless oil (1.7 g) which was purified by flash chromatography on silica eluting with 2:1 hexane:ethyl acetate to give the desired product as a white foamy solid (0.875 g, 1.55 mmol, 88%); as a 1:19 mixture of alpha:beta anomers.
Beta anomer:

m.p.=39-41° C., [α]_{D}=−24.9 (c=0.47, CHCl₃) ¹H NMR (CDCl₃, 400 MHz) δ ppm: 1.09 (s, 9H, tBu), 1.30 (s, 3H, CH₃), 1.47 (s, 3H, CH₃), 2.85 (d, J=3.1, 1H, OH), 3.16 (d, J=5.1, 1H, OH), 3.83 (dd, J=3.74, J=13.9, 1H, H-5′), 3.90 (dd, J=3.80, J=13.5, 1H, H-5′), 3.96 (s, 6H, 2×OMe), 4.27 (m, 2H), 4.35 (d, J=4.1, 1 H), 5.29 (s, 1 H, H-1′), 7.4 (m, 6H, Ph), 7.7 (m, 4 H, Ph), 8.38 (s, 1 H, H-6)

### Step 5

The preparation of: 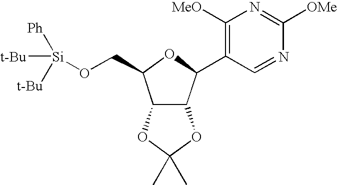
Triphenylphosphine (0.556 g, 2.12 mmol) was added to the product of step 4 (0.80 g, 1.42 mmol) in THF (anhydrous, 105 ml). The colourless mixture was cooled to 0° C. and diethyl azodicarboxylate¹ (0.35 ml, 2.12 mmol) was added dropwise. The yellow-orange mixture was stirred overnight at 4° C., then at room temperature for one hour, until no more starting material was observed by TLC. The mixture was concentrated and the orange oil (1.4 g) was purified by flash chromatography eluting with 4:1 hexane:ethyl acetate to give the desired product as a pure colourless oil (0.635 mg, 1.16 mmol, 82%).

[α]_{D}=+10.5 (c=0.55 CHCl₃) ¹H NMR (CDCl₃, 400 MHz) δ ppm: 1.06 (s, 9H, tBu), 1.35 (s, 3H, CH₃), 1.60 (s, 3H, CH₃), 3.83 (dd, J=4.6, J=11.1, 1H, H-5′), 3.88 (dd, J=3.95, J=11.23, 1H, H-5′), 3.94 (s, 3H, OMe), 3.99 (s, 3H, OMe), 4.13 (m, 1H), 4.65 (dd, 1H, J=4.2, 6.4), 4.72 (dd, 1H, J=4.6, J=6.5), 4.98 (d, 1H, J=4.1, H-1′), 7.3-7.4 (m, 6H, Ph), 7.6-7.8, (m, 4H, Ph), 8.31 (s, 1H, H-6).

### Step 6

The preparation of: 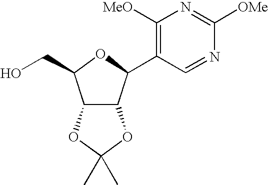
In a 100 ml round bottomed flask, the product of step 5 (0.940 g, 1.709 mmol) was dissolved in dry THF (60 ml). A 1M solution of tetrabutylammonium fluoride in THF (1.96 ml, 1.96 mmol) was added dropwise, at 0° C., then stirred for 45 minutes at 0° C. and 15 minutes at room temperature. The solution was then concentrated (40° C., 10 mmHg) to give an oil that was purified by flash chromatography on silica eluting with 5:5 hexane:ethyl acetate to give the title compound quantitatively as a colorless oil, as a 1:19 mixture of alpha:beta anomers.

[α]_{D}²⁰=−14.6° (c 0.57, CHCl₃) I.R. (Neat) ν cm⁻¹: 3397, 2990, 2938, 1606, 1573, 1473, 1400, 1213, 1075, 756. ¹H NMR (CDCl₃, 400 MHz) δ ppm: 1.34 (s, 3H, CH₃), 1.59 (s, 3H, CH₃), 2.35 (dd, 1H, J=3.1, J=8.6, OH), 3.75 (m, 1H, H-5′), 3.87 (m, 1H, H-5′), 3.98 (s, 3H, OMe), 4.02 (s, 3H, OMe), 4.13 (m, 1H, H-4′), 4.7-4.9 (m, 3H, H-1′, H-2′, H-3′), 8.20 (s, 1H, H-6). ¹³C NMR (CDCl₃, 400 MHz) δ ppm: 25.37, 27.45, 54.07, 54.85, 62.46, 81.37, 81.96, 84.10, 84.29, 111.99, 114.49, 157.88, 165.33, 168.69.

### Step 7

The preparation of: 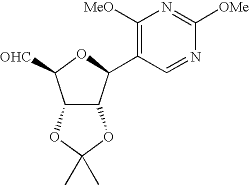
Oxalyl chloride (186 μL, 2.132 mmol) was added to dry dichloromethane (5 ml) and the solution cooled to −78° C. under argon, and DMSO (186 μL, 2.132 mmol) added dropwise. After 5 minutes, the product of step 6 (0.582 g, 1.867 mmol) was added in dry dichloromethane (2 ml). After one hour at −78° C., triethylamine (1.30 ml, 95 mmol) was added dropwise and the solution was allowed to stay at room temperature. Water (25 ml) was added, and the mixture was extracted with dichloromethane. The combined organic phases were washed with 1% hydrochloric acid (6 ml), water (10 ml) and brine (10 ml). The organic phase was dried over sodium sulphate, filtered and concentrated to afford the desired product as a white solid (0.503 g, 1.625 mmol, 87%).

¹H NMR (CDCl₃, 400 MHz) δ ppm: 1.37 (3H, CH₃), 1.59 (3H, CH₃), 3.90 (3H, CH₃), 3.99 (3H, CH₃), 4.48 (s, 1H), 4.81 (d, J=5.6), 5.03 (d, J=4.1), 5.14 (s, 1H), 8.18 (1H, s), 9.55 (s, 1H).

### Step 8

The preparation of: 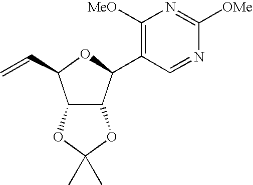
An oven dried 100 ml round bottomed flask was charged with Ph₃PCH₃⁺ Br⁻¹ (0.80 g, 2.23 mmol) and heated to 140° C. under vacuum (1 mm Hg) overnight. The flask was then put under an argon atmosphere and dry diethyl ether (22 ml) was added. At room temperature potassium tert-butoxide (2.23 ml of 1M solution in THF, 2.23 mmol,) was added. It was stirred at room temperature for one hour, and at −40° C., the product of step 7 (1.625 mmol) in dry THF (3 ml) was added dropwise. After two hours at −40° C., the solution was stirred at 0° C. overnight. Then at 0° C., a saturated solution of ammonium chloride (20 ml) was added. The aqueous phase was extracted with diethyl ether (3×40 ml). The combined organic phase were dried over sodium sulphate, filtered and concentrated. The crude oil was purified by flash chromatography under silica eluting with 8:2, hexane:ethyl acetate to give the desired product as a colourless oil (0.463 g, 1.218 mmol, 75%).

[α]_{D}²⁰=+29.5° (c 1, CHCl₃). I.R. ν cm⁻¹: 2989, 1606, 1572, 1472. ¹H NMR (CDCl₃, 400 MHz) δ ppm: 1.32 (s, 3H, CH₃), 3.96 (s, 3H, OMe), 3.98 (s, 3H, OMe), 4.35 (t, 1H, J=5.8), 4.50 (t, J=5.9, 1H), 4.68 (dd, 1H, J=3.6, J=6.5), 4.95 (d, 1H, J=3.2, H-1′), 5.23 (d, 1H, J=10.0, H-6′), 5.39 (d, 1H, J=17.2, H-6′), 5.92 (m, 1H, H-5′), 8.22 (s, 1H, H-6). ¹³C NMR (CDCl₃, 400 MHz) δ ppm: 25.47, 27.44, 53.97, 54.73, 80.04, 84.82, 85.15, 85.24, 112.77, 114.68, 117.57, 135.13, 156.88, 165.11, 168.51. HRMS GAB) Obtain 3081381 Calc 308.1372

### Step 9

The preparation of: 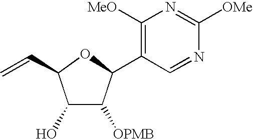
The product from step 8 (1.14 g, 3.70 mmol) was dissolved in 70% aqueous acetic acid (130 ml) and the solution was refluxed for one hour. After cooling, the solution was concentrated and the oil was co-evaporated twice with toluene (20 ml). The product and dibutyltin oxide (1.14 g, 5.6 mmol) in dry toluene (170 ml) were heated under reflux with removal of water during two hours. It was then concentrated, caesium fluoride (1.19 g, 7.8 mmol) was added and the flask was put under vacuum (2 mm Hg) for two hours. Tetrabutylammonium iodide (0.233 g, 0.631 mmol) and dry DMF (50 ml) and finally p-methoxybenzyl chloride (511 μL, 4.5 mmol) were added. The mixture was stirred for 36 hours at room temperature, poured into a saturated solution of sodium bicarbonate (150 ml) and water (150 ml). It was extracted with diethyl ether (5×200 ml). The combined organic phases were dried over sodium sulphate, filtered through celite and concentrated. The oil was purified by flash column chromatography to give the desired compound as a white solid (0.502 g, 35%, 1.29 mmol),

m.p.=74° C. [α]_{D}²⁰=+23.8° (c 0.5, CHCl₃). I.R. (KBr) ν cm⁻¹: 2970, 2870, 1602, 1574. NMR ¹H (CDCl₃, 400 MHz) δ ppm: 2.75 (b, 1H, OH), 3.80 (s, 3H, OMe), 3.87 (m, 1H, H-3), 3.93 (dd, 1H, J=2.8, J=5.5, H-2′), 3.98 (s, 3H, OMe), 4.00 (s,3H, OMe), 4.24 (dd, 1H, J=6.5, J=7.5, H-4′), 4.53 (d, 1H, J=1.3, CH₂Ar), 4.70 (d, 1H, J=11.3, CH₂Ar), 5.05 (d, 1H, J=2.6, H-1′), 5.28 (d, 1H, J=10.0, H-6′), 5.44 (d, 1H, J=17.1, H-6′), 5.9-6.05 (m, 1H, H-5′), 6.86 (d, 2H, J=8.6, Ar), 7.22 (d, 2H, J=8.6, Ar), 8.21 (s, 1H, H-6). HRMS (MAB) obtained 388.1645; calcd 388.1634

### Step 10

The preparation of 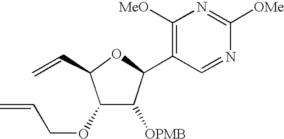
To a stirred mixture of dry DMF (1.6 ml) and sodium hydride (0.067 g, 1.67 mmol, 60% in a mineral oil) was added dropwise a solution of the product of step 9 (0.334 g, 0.861 mmol) in dry DMF (4.6 ml) and allyl bromide (161 μL, 1.86 mmol) at 0° C. When the addition was completed, the solution was stirred at room temperature under argon for two hours. A few drops of methanol were added at 0° C. and the solution was poured into water (50 ml). The aqueous phase was extracted with diethyl ether (4×50 ml), the combined organic phases were dried over sodium sulphate, filtered and concentrated. The oil was purified by flash column chromatography to give desired product as a colourless oil (0.344 g, 0.80 mmol, 93%).

[α]_{D}²⁰=+37.7° (c 0.97, CHCl₃) I.R. ν cm⁻¹: 2956, 1603, 1571, 1514. ¹H NMR (CDCl₃, 400 MHz) δ ppm: 3.62 (dd, 1H, J=4.8, J=7.5, H-3′), 3.77 (s, 3H, OMe), 3.92 (m, 3H, H-2, CH₂allyl), 3.97 (s, 6H, 2×OMe), 4.52 (t, 1H, J=7.5, H-4), 4.59 (d, 1H, J=11.9, CH₂Ar), 4.65 (d, 1H, J=11.9, CH₂Ar), 5.15-5.3 (m, 5H, H-1, 3×CH ethylenic), 5.45 (d, 1H, J=17.1, CH ethylenic), 5.75-6.0 (m, 2H, CH ethylenic), 6.82 (d, 2H, J=8.6, Ar), 7.23 (d, 2H, J=8.6, Ar), 8.21 (s, 1H, H-6). ¹³C NMR (CDCl₃, 400 MHz) δ ppm: 53.81, 54.67, 55.10, 71.20 (2C), 78.09, ,79.43, 80.81, 81.28, 113.48, 113.54, 117.28, 117.94, 129.31, 129.74, 134.14, 135.87, 156.34, 159.14, 164.81, 167.85.

### Step 11

The preparation of: 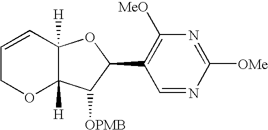
The product from step 10 (0.344 g, 0.80 mmol) was added to dry dichloromethane (160 ml) and argon was bubbled for 20 minutes into the solution. Then the Grubbs catalyst, [(cyclohexyl)₃P]₂Cl₂Ru═CHPh (0.039 g, 0.047 mmol) was added, and a slight flow of argon was applied. The solution was refluxed for 5.5 hours. When the reaction was complete, the mixture was concentrated to give a brown oil, which was purified by flash chromatography eluting with 8:2 hexane:ethyl acetate, to give the desired product as a white crystalline solid (0.286 g, 0.71 mmol, 89%), m.p. 80° C.

[α]_{D}²⁰=−33.18° (c 1.1, CHCl₃) I.R. ν cm⁻¹: 2956, 1603, 1602, 1575. ¹H NMR (CDCl₃, 400 MHz) δ ppm: 3.42 (dd, 1H, J=4.5, J=8.9, H-3′), 3.77 (s, 3H, OMe), 3.93 (m, 1H, H-2′), 3.95 (s, 3H, OMe), 3.96 (s, 3H, OMe), 4.40 (m, 2H, H-7′, H-7″), 4.54 (m, 1H, H-4′), 4.65 (d, 1H, J=11.9, CH₂Ar), 4.74 (d, 1H, J=11.9, CH₂Ar), 5.11 (m, 1H, H-1′), 5.69 (dm, 1H, J=10.3, H-5′), 6.28 (d, 1H, d=9.7, H-6′), 6.85 (d, 2H, J=8.7, Ar), 7.28 (d, 2H, J=8.7, Ar), 8.18 (s, 1H, H-6) 13C NMR (CDCl₃, 400 MHz) δ ppm: 53.87, 54.69, 55.11, 68.61, 71.28, 71.63, 78.78, 79.49, 81.72, 113.42, 113.62, 127.14, 127.40, 129.14, 130.01, 156.11, 159.06, 164.84, 167.55. HRMS (MAB) obtained 401.1699; calcd 401.1710

### Step 12

The preparation of: 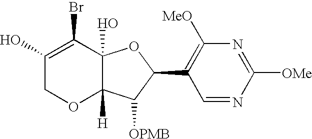
The product from step 11 (0.133 g, 0.33 mmol) was dissolved in THF (7.4 ml) and water (7.4 ml). N-bromosuccinimide (0.070 g, 0.39 mmol) was added to the reaction mixture, which was stirred vigorously for two hours in the dark. Then the mixture was poured into water (50 ml) (with one crystal of sodium thiosulfate) and extracted with diethyl ether (5×25 ml). The combined organic phase was dried over sodium sulphate and evaporated to give the crude desired bromo-alcohol product as a white solid, which was used for the next reaction without further purification.

### Step 13

The preparation of: 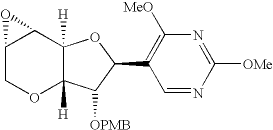
The crude bromo-alcohol product from step 12 was dissolved in THF (14 ml) and sodium hydroxide (3.55 ml, 1M in water) was added. The solution was refluxed for 45 minutes, then poured into water (50 ml). The solution was extracted with diethyl ether (4×40 ml), the combined organic phase were dried over sodium sulphate and evaporated to give the crude desired epoxide, which was used without further purification.

### Step 14

The preparation of: 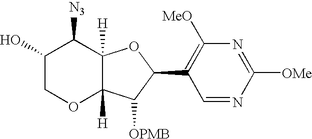
The epoxide product from step 13 was dissolved in methoxyethanol (30 ml) and sodium azide (0.327 g, 5.02 mmol) was added. The solution was heated at 126° C. for 1.25 hours. After cooling, the solution was poured into brine (50 ml) and extracted with diethyl ether (4×20 ml). The combined organic phase were dried over sodium sulphate and concentrated. The oil was purified by chromatography on silica (7:3 hexane/ethyl acetate) to give the desired product as a white amorphous solid (0.063 mg, 0.132 mmol, 41%), m.p. 119° C.

[α]_{D}²⁰+93.1° (c 0.98, CHCl₃). I.R. ν cm⁻¹: 3400, 2915, 2105 (N₃), 1604, 1572. ¹H NMR (CDCl₃, 400 MHz) δ ppm: 3.40 b, 1H, OH), 3.58(dd, 1H, J=4.7, J=10.0, H-3′), 3.68 (m, 1H, H-6′), 3.73 (d, J=12.7, 1H, H-7), 3.78 (s, 3H, OMe), 3.89 (m, 2H, H-2′, H-7′), 3.97 (s, 6H, 2×OMe), 4.33 (m, 2H, H-5′, H-4′), 4.67 (s, 2H, CH₂Ar), 5.10 (m, 1H, H-1′), 6.86 (d, 2H, J=8.7, Ar), 7.27 (d, 2H, J=8.7, Ar), 8.33 (s, 1H, H-6) ¹³C NMR (CDCl₃, 400 MHz) δ ppm: 53.87, 54.77, 55.12, 61.62, 68.26, 69.25, 71.41, 73.32, 74.16, 79.80, 80.25, 113.26, 113.66, 129.24, 129.65, 155.76, 159.18, 164.83, 167.35. HRMS (FAB) MH⁺° Calcd 460.1832; Found 460.1842

### Step 15

The preparation of: 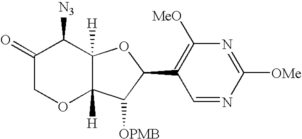
The product of step 14 (0.06 g, 0.129 mmoL) was dissolved in dry dichloromethane (DCM) (3 ml) under an argon atmosphere, and [1,1,1-tris(acetyloxy)-1,1-dihydro-1,2-benziodoxol-3-(1H)-one] (the Dess-Martin periodinane) (0.133 g, 0.42 mmol) was added. After 3.5 hours, a saturated solution of sodium bicarbonate (2.5 ml) and sodium bisulphite (2.5 ml) were added. The mixture was stirred for 10 minutes, DCM (25 ml) was added and the organic phase was lo separated and washed with saturated sodium bicarbonate (2×10 ml). After drying over sodium sulphate, the solution was evaporated to give the crude desired ketone product as an oily residue.

### Step 16

The preparation of: 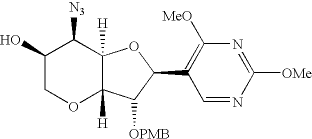
The crude product of step 15 was dissolved in dry methanol (6 ml), treated with sodium borohydride (0.050 g, 1.32 mmol) at 0° C. and the solution was stirred at room temperature for 10 minutes. The solution was concentrated under vacuum without heating. It was then dissolved in ethyl acetate (20 ml) and washed with water (20 ml). The organic phase was separated and the aqueous layer was extracted with ethyl acetate (2×20 ml). The combined organic phase were dried over sodium sulphate, and evaporated to give the crude desired hydroxy product as an oil.

### Step 17

The preparation of: 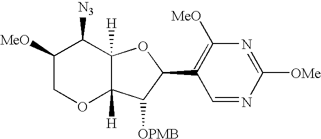
To a stirred mixture of DMF (0.5 ml) and sodium hydride (0.019 g, 60%, 0.49 mmol) at 0° C., a solution of the crude product from step 16, methyl iodide (38 μL, 5.9 mmol) in DMF (2 ml) was added dropwise. The mixture was then left at room temperature for 2 hours, a few drops of methanol were added at 0° C. and the solution was poured into cold water (20 ml). It was then extracted with ether (5×10 ml). The combined organic phase were dried over sodium sulphate and evaporated. The oil was purified by flash column chromatography under silica (8:2 hexane:ethyl acetate) to give the desired product as a white powder (0.056 g, 0.121 mmol, 93%, 3 steps), m.p. 89° C.

[α]_{D}²⁰+60.56° (c 0.88, CHCl₃). I.R. ν cm⁻¹: 2913, 2105 (N₃), 1602, 1572. ¹H NMR (CDCl₃, 400 MHz) δ ppm: 3.47 (s, 3H, OMe), 3.61 (m, 2H, H-7′, H-6′.), 3.76 (m, 4H, OMe, H-3′), 3.85 (d, J=4.63, 1H, H-2′), 3.96 (s, 3H, OMe), 3.97 (s, 3H, OMe), 4.01 (dd, J=10.04, J=2.91, 1H, H-4′), 4.67 (m, 3H, CH₂Ar, H-5′), 5.16 (s, 1H, H-1′), 6.86 (d, 2H, J=8.6, Ar), 7.26 (d, 2H, J=8.6, Ar), 8.44 (s, 1H, H-6) ¹³C NMR (CDCl₃, 400 MHz) δ ppm: 53.86, 54.76, 55.13, 57.13, 57.44, 59.72, 65.69, 71.17, 74.02, 75.01, 76.41, 78.89, 81.54, 113.31, 113.66, 129.76, 156.02, 159.10, 164.88, 167.23. HRMS (FAB) MH⁺ Calcd. 474.1988; Found 474.2002

### Step 18

The preparation of: 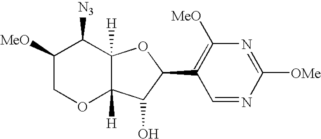
The product of step 17 (0.043 g, 0.09 mmol) was dissolved in DCM (8 ml), then water (0.8 ml) and 2,3-dichloro-5,6-dicycano-1,4-benzoquinone (DDQ) (0.080 g, 0.35 mmol) were added. After one hour, saturated sodium bicarbonate (30 ml) was added, the organic layer was separated and the aqueous phase was extracted with ethyl acetate (3×10 ml). The combined organic phases were washed with water (10 ml) and brine (10 ml). After drying over sodium sulphate and concentrating, the oil was purified by flash chromatography (DCM then DCM:methanol, 95:5) to give the desired compound as a colourless oil (0.027 g, 0.076 mmol, 84%).

[α]_{D}²⁰+93° (c 0.3, CHCl₃). IR (Neat) ν cm⁻¹: 3540, 2920, 2105, 1603, 1574. ¹H NMR (CDCl₃, 400 MHz) δ ppm: 3.49 (s, 3H, OMe), 3.57 (ddd, 1H, J=2.91, J=4.85, J=10.4, H-6′.), 3.65 (t, J=10.4, 1H, H-7′ax), 3.76 (dd, J=4.64, J=9.8, 1H, H-3′), 3.94 m(m, 2H, H-4′, H-7′eq), 3.98 (m, 4H, OMe), 4.02 (s, 3H, OMe), 4.10 (d, J=4.6, 1H, H-2′), 4.67 (m, 2H, CH₂Ar), 5.06 (s, 1H, H-1′), 8.41 (s, 1H, H-6) ¹³C NMR (CDCl₃, 400 MHz) δ ppm: 54.06, 54.79, 57.43, 59.40, 65.67, 73.05, 73.65, 74,67, 76.28, 83.53, 112.91, 155.77, 164.41, 167.61. HRMS (FAB) MH⁺ Calcd. 353.1335; Found 353.1341

### Step 19

The preparation of: 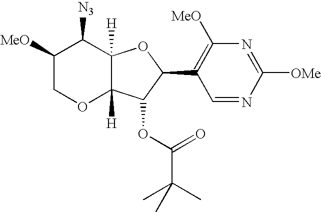
The product from step 18 (0.033 g, 0.0926 mmol) was dissolved in dry pyridine (1.1 ml). To this solution was added 4-dimethylaminopyridine (DMAP) (16 mg, 0.13 mmol), triethylamine (31 μL, 0.265 mmol) and pivaloyl chloride (80 μL, 0.649 mmol) and the solution was stirred for 36 hours at room temperature. It was then concentrated, the residue was dissolved ethyl acetate (20 ml) and the organic layer was washed with aqueous 1% hydrochloric acid (5 ml), followed by a saturated solution of sodium bicarbonate (5 ml) and brine (5 ml). After drying over sodium sulphate it was evaporated to give the crude desired pivaloyl compound as an oil.

### Step 20

The preparation of: 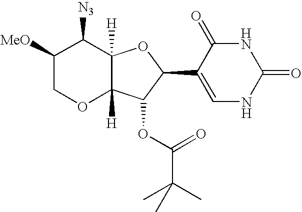
The crude product from step 19 was dissolved in dry acetonitrile (0.5 ml). Dry sodium iodide (0.032 g, 0.21 mmol), and trimethylsilyl chloride (29 μL, 0.21 mmol) were added and the solution was stirred at room temperature for 16 hours. Several drops of a 10% sodium metabisulphite solution were added until a colourless solution was obtained. Then a saturated solution of sodium bicarbonate (10 ml) was added, the aqueous layer was extracted with ethyl actetate (5×10 ml). The combined organic phases were dried over sodium sulphate and evaporated to give an oily residue which was purified by flash chromatography to give the desired product as a colourless oil (0.016 g, 42%, 0.0388 mmol).

[α]_{D}²⁰+100.75° (c 0.4, CHCl₃). ¹H NMR (CDCl₃, 400 MHz) δ ppm: 1.22, (s, 9H, tBu), 3.46 (s, 3H, OMe), 3.47 (ddd, 1H, J=2.9, J=4.0, J=10.7, H-6′.), 3.59 (t, J=10.7, 1H, H-7′ax), 3.75 (dd, J=2.7, J=10.0, 1H, H-4′), 3.86 (m, 2H, H-3′, H-7′eq), 4.65 (s, 1H, H-5′), 4.91 (s, 1H, H-1′), 5.31 (d, 1H, J=4.7, H-2′), 7.55 (s, 1H, H-6), 10.00 (br, 2H, 2×NH). ¹³C NMR (CDCl₃, 400 MHz) δ 26.97, 38.70, 57.38, 59.07, 65.55, 72.56, 72.81, 75.19, 76.35, 80.87, 111.63, 138.82, 152.17, 162.47, 171.14. HRMS (FAB) MH⁺ Calcd. 410.1675; Found 410.1693

### Step 21

Preparation of: 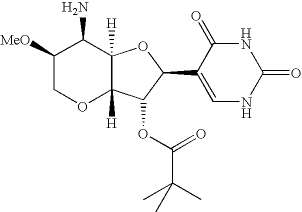
The product of step 20 (0.016 g, 0.0388 mmol) was dissolved in dry THF (3 ml) and argon was bubbled in the solution for 10 minutes. Water (3 μL) and trimethylphosphine (44 μL, 1M solution in toluene, 0.044 mmol) were added. After 5 minutes at room temperature, the solution was refluxed for 30 minutes, then concentrated and held under vacuum for one hour to give the crude desired amine product, which was used without further purification.

### Step 22

The preparation of: 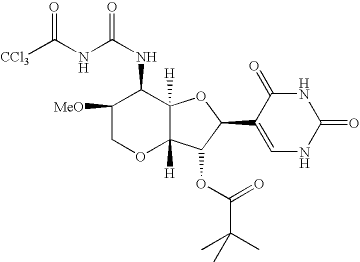
The crude product of step 21 was dissolved in dry DCM (4 ml) and trichloroacetylisocyanate (5 ml, 0.041 mmol) was added. After 30 minutes, the solution was concentrated to give the crude desired trichloroacetyl urea as an oil, which was used without further purification.

### Step 23

The preparation of malayamycin A 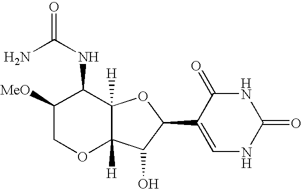
The crude product from step 22 was dissolved in methanol (1 ml), 40% methylamine in water (2 ml) was added and the solution was stirred for 52 hours. Concentration gave a solid that was purified by flash chromatography (9:1 DCM:methanol) to give pure malayamycin A as a white solid (0.008 g, 0.0233 mmol, 60%), m.p. 158° C. (dec).

[α]_{D}²⁰+120° (c 0.19, MeOH) (authentic sample [α]_{D}²⁰+126° (c 0.36, MeOH)) ¹H NMR (D₂O, 400 MHz) identical to the authentic sample δ ppm: 3.30 (s, 3H, OMe), 3.38 (t, 1H, J=10.7, H-7ax′.), 3.51 (dd, J=10.7, J=5.1, 1H, H-3′), 3.69 (ddd, J=5.2, J=3.5, J=10.7, 1H, H-6′), 3.85 (dd, J=3.5, J=11.8, 1H, H-7eq′), 3.93 (dd, J=10.7, J=5.4, 1H, H-6′), 4.16 (d, 1H, J=2.1, H-2′), 4.74 (s, 1H, H-1′), 4.82 (s, 1H, H-5′), 7.24 (s, 1H, H-6).

### EXAMPLE 2

In this Example, data are provided for key intermediates in the preparation of 6-epi-malayamycin A.

### Intermediate 1

Intermediate 1 is prepared from the product of Step 14 in Example 1, using the same procedure as given in Step 17 for malayamycin A in Example 1.

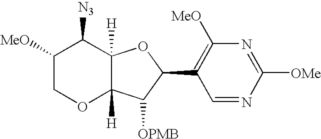

M.p. 100° C. [α]_{D}²⁰+53.5° (c 0.88, CHCl₃). I.R.(KBr) ν cm⁻¹: 2925, 2109 (N₃), 1602, 1571. ¹H NMR (CDCl₃, 400 MHz) δ ppm: 3.34 (s, 1H, H-6′), 3.45 (s, 3H, OMe), 3.67 (m, 2H, H-7′, H-3′.), 3.79 (m, 3H, OMe), 3.85 (d, J=4.51, 1H, H-2′), 3.96 (s, 3H, OMe), 3.98 (s, 3H, OMe), 4.08 (d, J=12.9, 1H, H-7′), 4.35 (dd, 1H, J=3.09, J=10.01, H-4′), 4.48 (s, 1H, H-5′), 4.63 (d, J=12.05, 1H, CH₂Ar), 4.77 (d, J=12.05; 1H, CH₂Ar), 5.07 (s, 1H, H-1′), 6.86 (d, 2H, J=8.35, Ar), 7.29 (d, 2H, J=8.35, Ar), 8.44 (s, 1H, H-6) ¹³C NMR (CDCl₃, 400 MHz) δ ppm: 54.36, 55.28, 55.67, 57.75, 60.01, 66.43, 71.84, 74.15, 74.87, 77.75, 79.85, 81.10, 114.03, 114.11, 129.63, 130.68, 156.33, 159.55, 165.38, 168.00. HRMS (FAB) M⁺ Calcd. 473.1910; Found 474.1910

### Intermediate 2

Intermediate 2 is prepared from Intermediate 1 using the same procedure given in Step 18 for malayamycin A in Example 1.

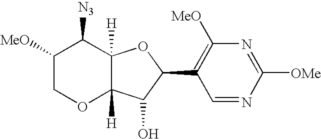

[α]_{D}²⁰+78.9° (c 0.3, CHCl₃). IR (Neat) ν cm⁻¹: 3538, 2918, 2109, 1602, 1571. ¹H NMR (CDCl₃, 400 MHz) δ ppm: 2.60 (br, 1H, OH), 3.33 (m, 1H, H-6), 3.44 (s, 3H, OMe), 3.64 (dd, 1H, J=3.11, J=10.13, H-3′.), 3.72 (dd, J=1.2, J=12.9, H, H-7′ax), 3.98 (m, 3H, OMe), 4.02 (s, 3H, OMe), 4.04 (d, J=12.09, 1H, H-7′eq), 4.14 (d, J=4.86, 1H, H-2′), 4.28 (dd, J=3.21, J=10.03, 1H, H-4′), 4.49 (m, H-5, 1H), 5.00 (s, 1H, H-1l), 8.32 (s, 1H, H-6) ¹³C NMR (CDCl₃, 400 MHz) δ ppm: 54.57, 55.30, 57.88, 58.65, 66.65, 73.92, 74.07, 74.21, 77.29, 83.03, 113.60, 156.19, 165.49, 168.33. HRMS (FAB) MH⁺ Calcd. 353.1335; Found 353.1334

### Intermediate 3

Intermediate 3 is prepared from Intermediate 2 using the same procedures given in Steps 19 and then 20 for malayamycin A in Example 1.

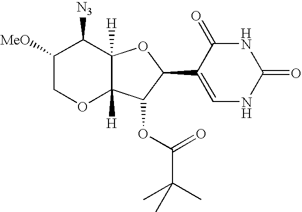

[α]_{D}²⁰+143° (c 0.53, CHCl₃). ¹H NMR (CDCl₃, 400 MHz) δ ppm: 1.23, (s, 9H, tBu), 3.29 (d, J=3.15, H-6, 1H), 3.37 (s, 3H, OMe), 3.59 (d, 1H, J=12.1, 1H, H-7′), 3.78 (dd, J=4.88, J=10.13, 1H, H-3′), 4.00 (d, 1H, J=13.2, H-7′eq), 4.15 (dd, J=3.24, J=10.12, 1H, H-4′), 4.43 (t, 1H, J=2.99, H-5), 4.82 (s, 1H, H-1′), 5.38 (d, J=4.93, H-2′), 7.44 (d, J=5.8, 1H, H-6), 11.02 (br, 2H, NH). ¹³C NMR (CDCl₃, 400 MHz) δ ppm: 26.94, 38.74, 56.60, 59.15, 64.87, 72.74, 73.12, 73.86, 76.71, 79.60, 111.70, 138.54, 152.19, 162.48, 177.55. HRMS (FAB) MH⁺ Calcd. 410.1675; Found 410.1678

6-Epi-malayamycin A

6-Epi-malayamycin A is prepared from Intermediate 3 using the same procedures given in Steps 21, 22, and then 23 for malayamycin A in Example 1.

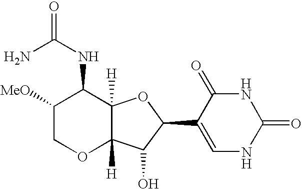

[α]_{D}²⁰+38.6° (c 0.3, MeOH). ¹H NMR (D₂O, 400 MHz) δ ppm: 3.37 (s, 3H, OMe), 3.43 (s, 1H, H-6′.), 3.49 (dd, J=10.9, J=5.13, 1H, H-3′), 3.71 (d, J=13.78, 1H, H-7′), 4.02 (m, 2H, H-7eq′+H-4′), 4.22 (d, 1H, J=2.5, H-2′), 4.52 (m, 1H, H-5′), 4.64 (s, 1H, H-1′), 7.33 (s, 1H, H-6).

### EXAMPLE 3

This Example illustrates the fungicidal properties of certain of the novel compounds of formula (I). The compounds were tested against a variety of foliar fungal diseases of plants. The technique employed was as follows.

Plants were grown on an artificial, cellulose based growing medium. The test compounds were individually diluted in reverse osmosis water to a final concentration of 100 ppm in water (that is, 1 mg of compound in a final volume of 10 ml) immediately before use. TWEEN 20 (at a final concentration of 0.05% by volume) was added with the water to improve retention of the spray deposit. TWEEN is a registered trade mark.

The compounds were applied to the foliage of the test plants by spraying the plant to maximum droplet retention.

These tests were carried out against Stagonospora nodorum (LEPTNO), Blumeria graminis f. sp. tritici (ERYSGT), and Puccinia triticina (PUCCRT) on wheat. Two replicates, each containing 3 plants were used for each treatment. The plants were inoculated with either a calibrated fungal spore suspension or a “dusting” with dry spores 6 hours or one day after chemical application.

After chemical application and inoculation, the plants were incubated under high humidity conditions (except those inoculated with Blumeria graminis f. sp. tritici) and then put into an appropriate environment to allow infection to proceed until the disease was ready for assessment. The time period between chemical application and assessment varied from six to nine days according to the disease and environment. However, each individual disease was assessed after the same time period for all compounds.

Assessments were carried out collectively on the plants in each replicate and averaged to give one result per replicate.

The disease level present (the percentage leaf area covered by actively sporulating disease) was assessed visually. For each treatment, the assessed values for all its replicates were meaned to provide mean disease values. Untreated control plants were assessed in the same manner. The data were then processed (see formula below) to calculate a PRCO (percentage Disease Reduction from Control) value.

### Banded Assessment Method and Calculation of PRCO Values

The mean disease values are banded in the manner shown below. If the disease level value falls exactly mid-way between two of the points, the result will be the lower of the two points.

| | | |
|---|---|---|
| 0 = | 0% | disease present |
| 1 = | 0.1-1% | disease present |
| 3 = | 1.1-3% | disease present |
| 5 = | 3.1-5% | disease present |
| 10 = | 5.1-10% | disease present |
| 20 = | 10.1-20% | disease present |
| 30 = | 20.1-30% | disease present |
| 60 = | 30.1-60% | disease present |
| 90 = | 60.1-100% | disease present |

An example of a typical banded calculation is as follows:
Mean disease level for treatment A=25%

Therefore banded mean disease level for treatment A=30
Mean disease level on untreated controls=85%

Therefore banded mean disease level on untreated controls=90

$$\begin{matrix}PRCO = 100 - \frac{\{ Banded mean disease level for treatment A \}}{\{ Banded mean disease level on untreated controls \}} \times 100 \\ = 100 - \frac{( 30 \times 100 )}{90} \\ = 66.7\end{matrix}$$

The PRCO is then rounded to the nearest whole number; therefore, in this particular example, the PRCO result is 67.

It is possible for negative PRCO values to be obtained.

PRCO results are shown below.

TABLE I

| COMPOUND | ERYSGT | PUCCRT | LEPTNO |
|---|---|---|---|
| NO. | 6 hour | 1 day | 1 day |
| (Table 1) | Protectant | Protectant | Protectant |
| 2 | 50 | 100 | 45 |
| 3 | 0 | 100 | 100 |

Key to Table I
ERYSGT = Blumeria graminis tritici
PUCCRT = Puccinia triticina
LEPTNO = Stagonospora nodorum

1. A process for the preparation of a compound of the general formula (I): 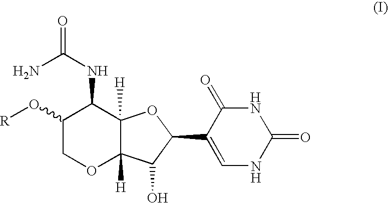
wherein R is H or C₁₋₄ alkyl, which comprises treating a compound of the general formula (II): 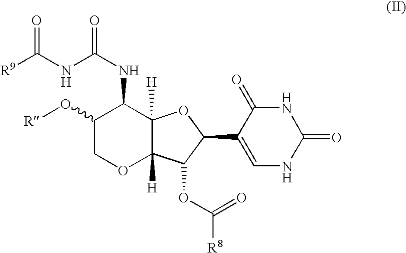
wherein R″ is R or R⁸CO, R is as defined above, R⁸ is C₁₋₈ alkyl or optionally substituted phenyl and R⁹ is optionally substituted C₁₋₈ alkyl or optionally substituted aryl, with an amine R′″NH₂ wherein R′″ is H or C₁₋₄ alkyl.

2. A process according to claim 1 wherein aryl is phenyl.

3. A process according to claim 1 wherein the optional substituents of aryl and phenyl are selected from the group consisting of C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, C₁₋₄ alkoxy(C₁₋₆)alkyl, C₃₋₆ cycloalkyl, C₃₋₆ cycloalkyl(C₁₋₄)alkyl, phenoxy, benzyloxy, benzoyloxy, cyano, nitro, —CONR^{a}R^{b}, —SO₂R^{a}, —OSO₂R^{a}, —COR^{a}, —CR^{a}═NR^{b} and —N═CR^{a}R^{b}, in which R^{a} and R^{b} are independently hydrogen, C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl.

4. A process according to claim 1 wherein R is H or C₁₋₄ alkyl, R″ is R or R⁸CO, R⁸ is C₁₋₆ alkyl and R⁹ is C₁₋₆ alkyl substituted with halo, especially chloro.

5. A process according to claim 1 wherein R is H or methyl, R″ is methyl or R⁸CO, R⁸ is tert-butyl and R⁹ is trichloromethyl.

6. The trans isomer of the compound of formula (I) according to claim 1 wherein R is CH₃ (6-epi-malayamycin A) and the trans isomer of the compound of formula (I) where R is H (6-epi-desmethylmalayamycin A).

7. The cis and trans isomers of the compound of formula (I) according to claim 1 wherein R is C₂₋₄ alkyl.

8. The intermediate compounds of the formulae (16) to (33) and (35) to (41): 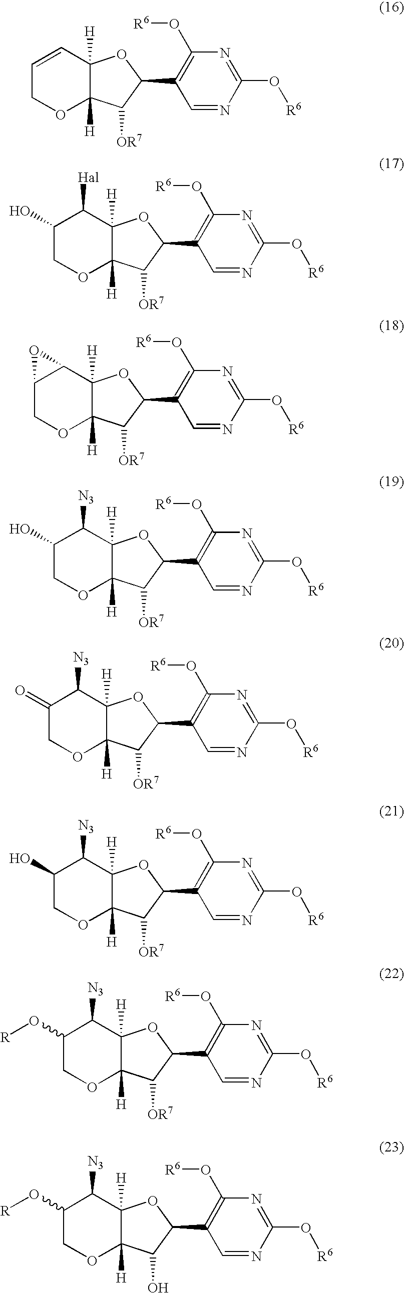 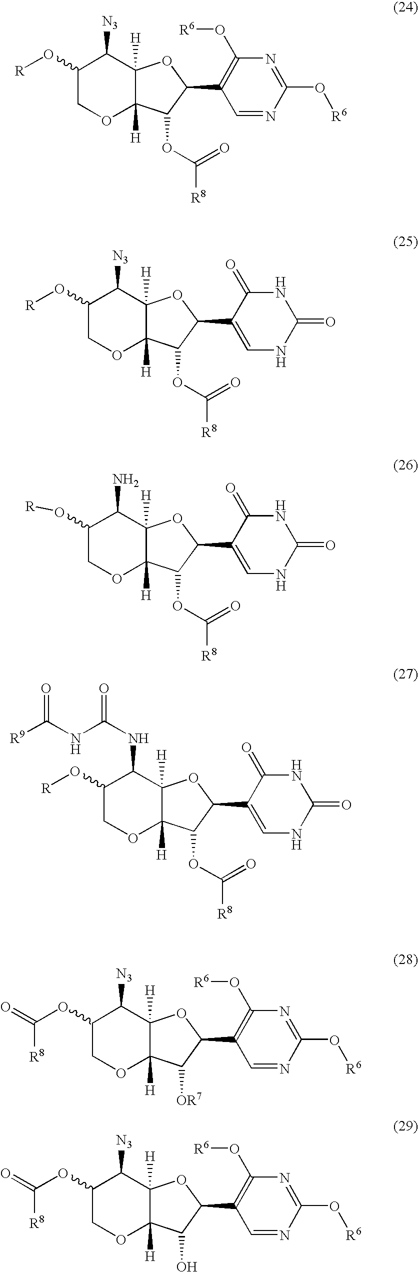 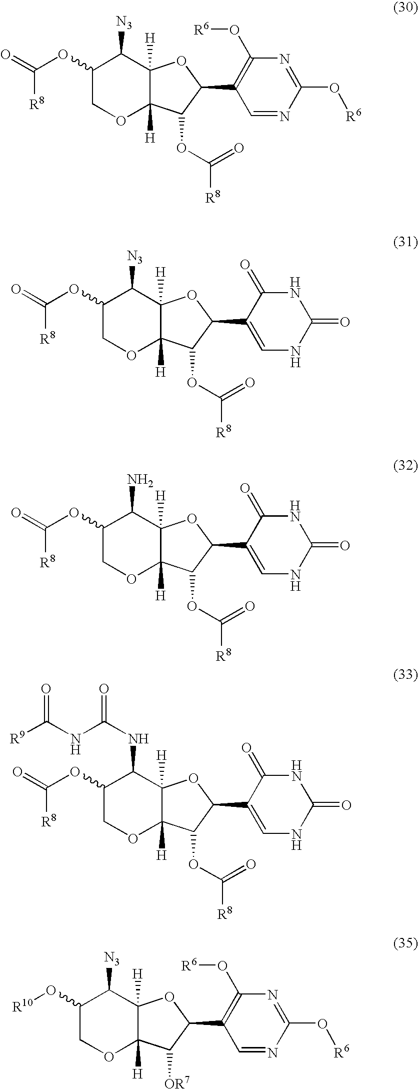 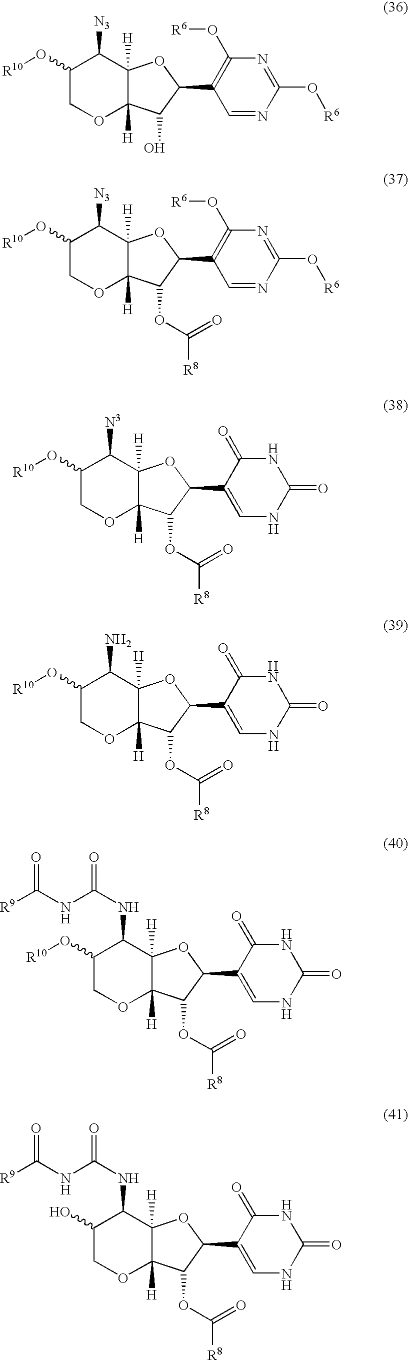
wherein R and R⁶ are independently C₁₋₄ alkyl, R⁷ is substituted benzyl, R⁸ is C₁₋₈ alkyl or optionally substituted phenyl, R⁹ is C₁₋₈ alkyl optionally substituted with halo or is optionally substituted aryl, R¹⁰ is a protecting group and Hal is halo.

9. A compound according to claim 8 wherein the substituents of benzyl and the optional substituents of phenyl are selected from the group consisting of C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)-alkoxy, C₁₋₆ alkylthio, halo(C₁₋₆)alkylthio, C₁₋₄ alkoxy(C₁₋₆)alkyl, C₃₋₆ cycloalkyl, C₃₋₆ cycloalkyl-(C₁₋₄)alkyl, phenoxy, benzyloxy, benzoyloxy, cyano, nitro, —CONR^{a}R^{b}, —SO₂R^{a}, —OSO₂R^{a}, —COR^{a}, —CR^{a}═NR^{b} and —N═CR^{a}R^{b}, in which R^{a}and R^{b} are independently hydrogen, C₁₋₄ alkyl, halo(C₁₋₄)alkyl, C₁₋₄alkoxy, halo(C₁₋₄)alkoxy, C₁₋₄ alkylthio, C₃₋₆ cycloalkyl, C₃₋₆ cycloalkyl-(C₁₋₄)alkyl, phenyl or benzyl.

10. A compound according to claim 8 wherein R and R⁶ are independently C₁₋₄ alkyl, R⁷ is substituted benzyl, R⁸ is C₁₋₆ alkyl, R⁹ is C₁₋₆ alkyl substituted with halo, especially chloro, R¹⁰ is (R¹¹)₃SiCH₂CH₂OCH₂ group where R¹¹ is C₁₋₄ alkyl and Hal is chloro, bromo or iodo.

11. A compound according to claim 8 wherein R and R⁶ are both methyl, R⁷ is 4-methoxybenzyl, R⁸ is tert-butyl, R⁹ is trichloromethyl, R¹⁰ is (CH₃)₃SiCH₂CH₂OCH₂ and Hal is bromo.
